(19) United States
(12) Patent Application Publication
(10) Pub. No.: US 2026/0092243 A1
(43) Pub. Date: Apr. 2, 2026

(54) MICROPHYSIOLOGICAL PLATFORM FOR DRUG ABSORPTION MODELING

(71) Applicant: Javelin Biotech, Inc., Woburn, MA (US)

(72) Inventor: Murat Cirit, Cambridge, MA (US)

(21) Appl. No.: 19/113,798

(22) PCT Filed: Sep. 19, 2023
(86) PCT No.: PCT/US2023/033174

§ 371 Date: Mar. 20, 2025

Related U.S. Application Data
(60) Provisional application No. 63/408,363, filed on Sep. 20, 2022.

(51) Int. Cl. C12M 3/00; C12M 1/00; C12M 1/34; G01N 33/50; G16C 20/70

(52) U.S. Cl. CPC C12M 21/08; C12M 29/24; C12M 41/26; C12M 41/34; G01N 33/5082; G16C 20/70

(57) ABSTRACT

Microphysiological platforms are configured for measuring kinetic rates relating to drug absorption. These kinetic rates include GI region-specific kinetic rates of drug absorption. The kinetic rates include bioavailability of the drug over time in various tissues. The kinetic rates include drug permeability in various tissues. The kinetic rates include measurements of microbial transformations that occur. The kinetic rates include measured effects of gastric emptying and transit times. The kinetic rates include measurements of fasting and fed states for various tissues.

### CLAIM OF PRIORITY

This application claims priority under 35 U.S.C. § 119(e) to U.S. Patent Application Ser. No. 63/408,363, filed on Sep. 20, 2022, the entire contents of which are hereby incorporated by reference.

### TECHNICAL FIELD

This disclosure generally relates to microfluidic devices. More specifically, this disclosure relates to drug absorption and bioavailability modeling platforms.

### BACKGROUND

It is desirable to develop a noninvasive method (e.g., sampling blood or urine, etc.) for monitoring drug bioavailability in humans. Generally, animal models are not accurate predictors of drug bioavailability in humans. There are many factors affecting drug absorption and bioavailability in humans. These factors relate to oral drug administration (if applicable), disintegration and drug dissolution, drug permeation in the intestinal membrane (if applicable), portal vein factors, hepatic vein factors, and factors relating to systemic circulation.

### SUMMARY

This disclosure describes microphysiological platforms for testing, by biomimetic in vitro simulation, drug absorption in humans. The microphysiological platforms model a plurality of human tissues and enable measurements of drug absorption and bioavailability in those tissues. Various factors affecting drug absorption are modeled from the testing and various outcomes are measured, including kinetic rates.

The microphysiological platforms are configured for measuring kinetic rates relating to drug absorption. These kinetic rates include GI region-specific kinetic rates of drug absorption. The kinetic rates include bioavailability of the drug over time in various tissues. The kinetic rates include drug permeability in various tissues. The kinetic rates include measurements of microbial transformations that occur. The kinetic rates include measured effects of gastric emptying and transit times. The kinetic rates include measurements of fasting and fed states for various tissues.

The microphysiological platforms are designed to model human tissues and in vitro environments based on a plurality of biomimetic parameters. These parameters can include flow rates of fluid (e.g., fluid medium that includes a drug dose), transit times, pH levels in tissues, oxygen tension, mucus thickness, microbiota development, and bile and digestive enzyme concentrations. The human tissues can relate to liver tissue, GI tissue, or other tissues that are related to drug absorption.

Microphysiological systems (MPSs) are configured to host human tissues in controlled environments. The MPSs can include a microenvironment simulating a GI tract. This gut MPS is used to monitor epithelial permeability. From experimentation using these MPSs, design parameters are obtained for drug absorption studies. These parameters include compartment volumes for chambers of the MPSs, tissue sizes and surface areas in the MPSs, flow paths in the MPSs, and media exchanges and oxygen circulation configurations in the MPSs. The MPS enable several parameters for drug absorption experiments to be tunable, including gastric emptying time, transit or recirculation time, mixing time, and microbiota density and culture configurations. Data gathered from adjusting these parameters enables development of a hardware MPS that enables control of tunable parameters and is calibrated to reduce or eliminate error in drug absorption measurements obtained from the tunable MPS.

The platform can enable one or more technical advantages. The MPSs described herein enable a quicker assessment of bioavailability compared to animal models, reduce inter-species differences, and provide detailed insights for complex mechanisms in drug absorption.

The one or more advantages described can be enabled by one or more aspects or embodiments of the platform.

The details of one or more embodiments of these systems and methods are set forth in the accompanying drawings and the description to be presented. Other features, objects, and advantages of these systems and methods are apparent from the description and drawings, and from the claims.

### BRIEF DESCRIPTION OF THE DRAWINGS

FIG. 1A is a block diagram of a microphysiological platform for drug absorption analysis.

FIGS. 1B-1D show example pump configurations for the microphysiological platform of FIG. 1A.

FIG. 2A shows an example of a seeding chamber for the microphysiological platform of FIG. 1A.

FIG. 2B shows an example of a seeding chamber for the microphysiological platform of FIG. 1A.

FIG. 3A is an image of a cell culture chamber and associated ports for the microfluidic platform for drug absorption analysis of FIG. 1A-1D and 2A-2B.

FIG. 3B includes images of cell cultures in the cell chambers of FIG. 3A.

FIG. 4 shows examples of cell cultures of the cell chambers of FIG. 3A.

FIG. 5 shows graphs including a comparison of Caco-2 cells under different culture conditions from static cultures.

FIGS. 6A-6C show example data of measured effects of luminal mucin, basal BSA & fasted state on propranolol permeability on static Caco-2 cultures.

FIG. 7 shows a diagram of an example cell culture chamber including apical and basal sub-chambers.

FIG. 8 shows example results of computational simulations for determining chip design and operational parameters including flow rate and media composition.

FIGS. 9A-9G show characterizations of cell cultures.

FIG. 10 is a graph showing TEER values for different tissue origins.

FIGS. 11A-11C show graphs indicating concentration values over time for iPSC-derived tissues.

FIG. 12 shows graphs indicating concentration values over time for iPSC-derived gut tissue, including apical-to-basal and basal-to-apical concentrations for prednisolone using for iPSC-derived gut tissue.

FIG. 13 is a block diagram of an example computer system used to provide computational functionalities associated with described algorithms, methods, functions, processes, flows, and procedures described in the present disclosure.

FIG. 14 is a diagram illustrating an example computer system configured to execute a machine learning model.

### DETAILED DESCRIPTION

This disclosure describes a microfluidic system including in vitro simulated environments for testing factors affecting drug absorption in human tissues. The microfluidic systems described in this disclosure enable testing of drug absorption in tissues present for oral drug administration, gastric emptying and intestinal transit, gut absorption and liver metabolism, and drugs in systemic circulation.

The factors describe how drug disintegration and dissolution occur. Factors that can affect drug disintegration in a person can include tissue age, food intake, posture, body mass index, stress levels, diseases present, formulation size and density, and whether drugs are concomitant. Drug dissolution rates are based on drug solubility, pKa, lipophilicity, particle size, diffusivity, gastrointestinal (GI) pH, viscosity and volume of GI fluid, formulation type (e.g., drug composition), and so forth. The microfluidic systems are configured to measure drug degradation, complexation, or precipitation. These are based on GI pH, crystal form of the drug, food components present, a presence of GI enzymes, and so forth.

The microfluidic systems are configured to measure drug permeation though the intestinal membrane. The microfluidic systems are configured to measure contributions to permeability from lipophilicity, molecular weight (MW) solubility, GI pH, unstirred water layer (UWL) thickness, and so forth. The MPS devices are configured for providing a platform, for intestinal metabolism based on presence or absence of Cytochromes P450 (CYPs) enzymes, Glutathione S-transferases (GSTs) enzymes, UDP-glucuronosyltransferase (UGTs) enzymes, superfamily of sulfotransferase (SULTs) enzymes, drug-drug interactions, and so forth. Generally, these enzymes are present in the GI tissue. MPS devices are a platform to house the GI tissue. The microenvironment (e.g., flow, microbes, etc.) enhances enzymatic functions (e.g., biomimicry) of the housed enzymes. The MPS is used to measure drug concentration changes, such as total drug consumption and permeability from apical to basal and from basal to apical. The intestinal drug permeation is measured by measuring influx transporters and efflux transporters (e.g., presence or absence and concentrations).

The MPS is configured to measure drug absorption in the GI tissue. The drug concentrations for the basal gut mimic drug concentration in the portal vein. The portal vein carries the drugs to liver for hepatic metabolism. The hepatic metabolism is determined by determining a presence or absence and function of CYPs, GSTs, UGTs, SULTs, protein binding, and so forth. The drug concentrations for the apical gut mimic drug concentrations in the GI lumen.

The microfluidic systems enable determination of drug kinetics. Specifically, microfluidic devices described in this specification enable determination of GI region specific kinetic rates, bioavailability, permeability, microbial transformations, effects of gastric emptying and transit times, kinetics for tissues in fasting and fed states, and so forth. These kinetic rates are determined from measurements of drug concentrations in the media including fluid medium samples extracted from the microfluidic devices. A data processing system determines the kinetic rates using one or more computational models.

The determined kinetic rates (e.g., using control tissues) enable tuning of several biomimetic parameters by adjusting the hardware design of the microfluidic circuit of the microfluidic devices. For example, the hardware is adjusted to tune the flow rate of the fluid medium, adjust transit time of the fluid medium, adjust a pH of the fluid medium, effect a particular oxygen tension in the fluid, tune a mucus thickness of the fluid medium, adjust presence and amount of microbiota in the circuit, and/or tune bile and digestive enzyme concentrations in the fluid medium or in cell chambers.

Once these factors are measured, models are developed to represent expected behavior in microenvironments. These models are used to develop microfluidic devices configured to test how new molecular entities (NCE) are absorbed. The modeling process includes developing drug kinetic profiles and parameters from the absorption microfluidic device, including bioavailability (Fa), clearance (CL), absorption (Papp), and so forth. The drug properties are associated with the performance of microfluidic devices. For example, in the MPS devices, pharmacokinetic (PK) properties of a drug are determined based on acquisition of fluid media samples. The determined PK properties are a function of chemical structure. The PK properties are used to determine chemical structure of the drug (e.g., SMILES, two-dimensional, three-dimensional, and so forth), and physicochemical properties (e.g., log). In some implementations, the drug ionization stage with respect to a microenvironment pH change is directly measured. Additionally, an intestinal metabolism is measured in the MPS device.

The drug property values and drug kinetic profiles measured from microfluidic devices are input into a machine learning algorithm for pharmacokinetic parameters, including bioavailability (Fa), clearance (CL), absorption (Papp), and intestinal metabolism.

The machine learning models are trained using the drug kinetic profiles and drug properties data. Both PBPK and QSP models receive the machine learning-corrected PK parameters to predict human pharmacokinetic profiles, oral drug absorption and bioavailability. The MPS is focused on the GI tract microenvironment to evaluate drug absorption and metabolism in gut tissues.

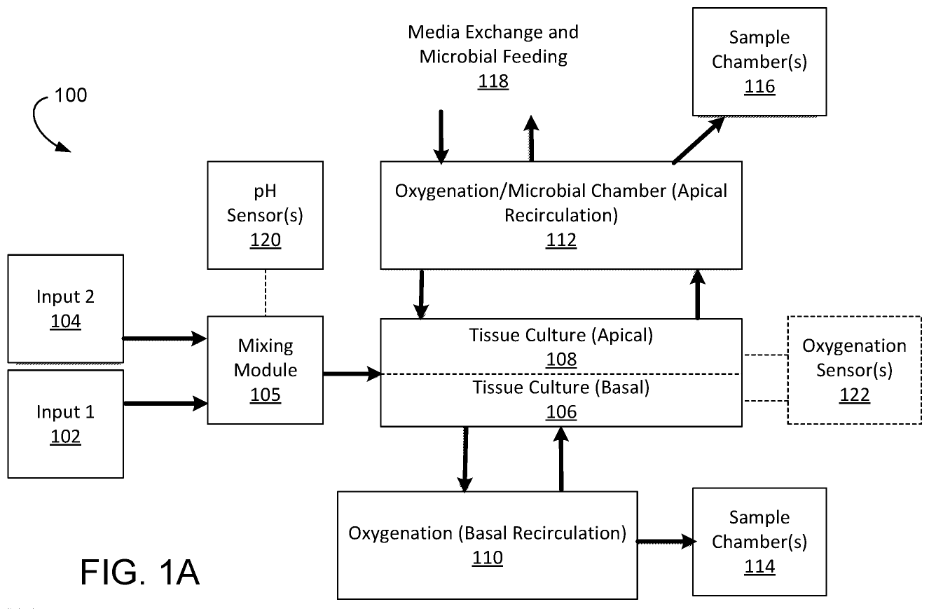

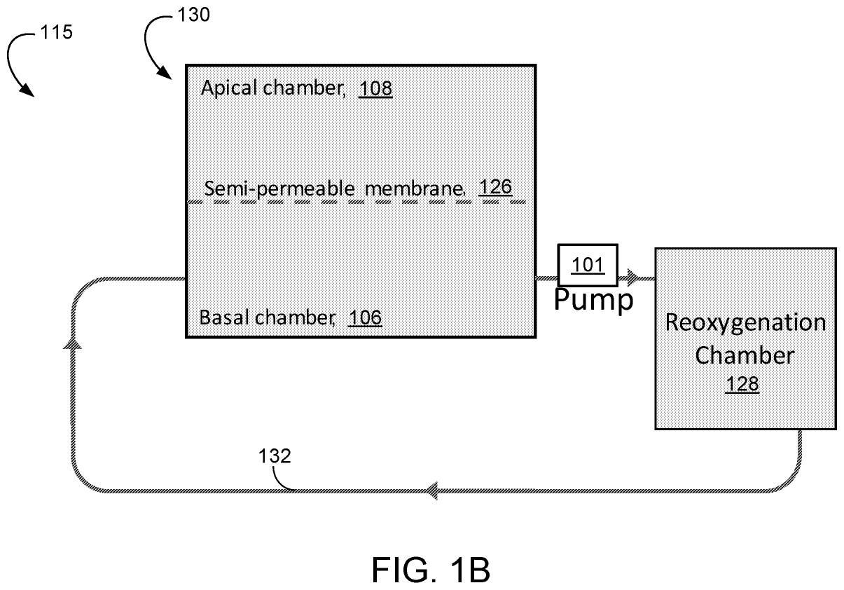

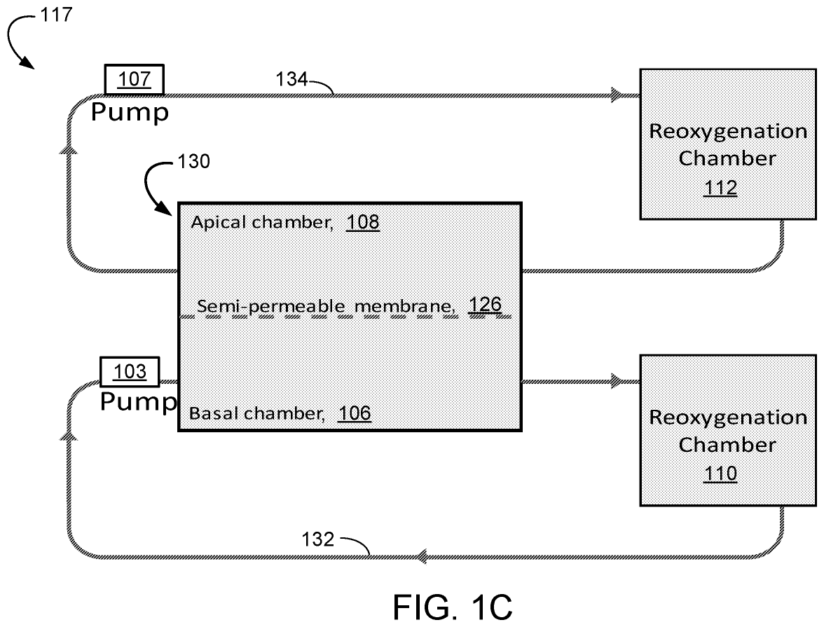

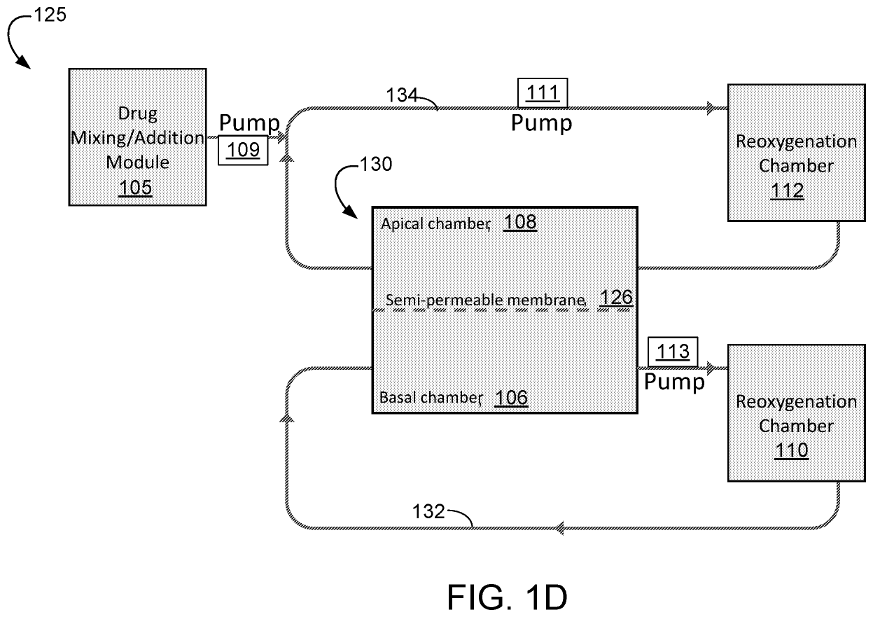

FIG. 1A is a block diagram illustrating an example hardware platform 100 for hosting one or more MPSs configured to measure drug absorption and/or bioavailability in human tissues. The platform 100 enables long-term tissue mono-& co-culture (e.g., longer than four weeks). The hardware platform 100 includes a cell chamber for both apical tissue cultures 108 and basal tissue cultures 106. The platform 100 includes a cell seeding port (not shown) for introducing these tissues to the cell chamber. The platform 100 includes media access ports or sample chambers 114, 116 for accessing samples of fluid media in the platform. Input port(s) 102, 104 enable media input into the fluid circuit of the platform 100. A fluid mixing chamber 104 mixes fluid media from inputs 102, 104 together. In some implementations, a pH sensor or sensors 120 monitor the pH of the fluid media once mixed and just prior to entry of the fluid into the cell chamber 106, 108. The cell chamber 106, 108 is also monitored by oxygenation sensor(s) 122 that measure oxygen levels in the fluid media of the cell chamber 106, 108. The oxygen level and pH of the fluid media are tuned to particular values in the cell chamber to simulate in vivo drug absorption. In some implementations, the cell chamber includes small intestine cells, large intestine cells, stomach cells, or a co-culture of GI tract tissue (e.g., in apical chamber 108) with liver tissue (e.g., in basal chamber 106).

A fluid recirculation and re-oxygenation chamber 110, 112 for each of the apical and basal tissues are each configured to mix fluid media in the platform 100 and add oxygen to the fluid media. A micro-pump (not shown) is included in a fluid circuit to circulate media through the cell chamber including tissues 106, 108 and the mixing and re-oxygenation chambers 110, 112. This fluid circuit can be configured to introduce substances (e.g., drugs or other substances) to the cell chamber to emulate functionality of in vivo human tests.

The platform 100 includes media exchange and microbial feeding chamber 118, which can be an optional chamber as shown by a dashed line in FIG. 1. The chamber 118 is configured to control bacterial growth (e.g., overgrowth) and fluid levels in the MPS. Bacterial culture density is adjusted in this chamber by addition new bacteria or removing (e.g., diluting) overgrowing culture. In some implementations, the microbial feeding chamber 118 is optionally included, and bacterial growth and fluid levels are controlled in chamber 112.

Chamber 118 differs from input chambers 102, 104 because chamber 118 has bacteria that metabolizes the drugs introduced to the system 100. Inputs 102 and 104 are physical mixing processes for introducing cells to the system, and no cellular (mammalian or bacterial) processes happen in these chambers. Additionally, inputs 102 and 104 are unidirectional pumping chambers (e.g., stomach to gut). Chamber 118 includes a bi-directional input/output path, including an input from apical gut and an output to the apical gut. The flow may be intermittent or continuous.

The platform 100 is configured for drug absorption modeling by enabling emulation of tissues in controlled environments. For example, the platform 100 enables measurement of GI region specific kinetic rates, intestinal metabolism, permeability, microbial transformations, effects of gastric emptying and transit times, kinetics for tissues in fasting and fed states, and so forth.

In some implementations, the platform 100 represents one or more regions of the GI tract. In some implementations, the platform 100 represents interconnected tissue systems. For example, the small and large intestine can be modeled together. For example, when modeling the small intestine, input 102 includes an active pharmaceutical ingredient (API) (pH ˜1.5) and input 104 includes a base, bile salts, or digestive enzymes. The cell culture is small intestinal cells having an apical pH of 6.0.

Using the MPS, several biomimetic parameters are applied to the tissue. For example, the MPS controls O₂ concentration to allow an anaerobic bacterial culture to grow for analysis. The MPS is configured to run with or without bacterial cultures. In another example, the MPS is configured to adjust parameters including a small intestinal permeability, a microbial transformation, a fast state, and a fed state. Similarly, for a large intestine, the input 102 is an API (pH ˜6.5) or apical samples from the small intestine. Input 104 includes bile salts and/or digestive enzymes. The digestive enzymes can include proteases, lipase, amylase, bile salts, and so forth. These are distinct from the drug metabolizing enzymes previously described.

The cell culture includes large intestinal cells with an apical pH of 6.0. The parameters that are adjusted include a large intestinal permeability, a microbial transformation, a fast state, and a fed state.

In some implementations, the platform 100 represents the stomach. In this example, platform 100 input 102 includes API, while input 104 is either empty or base. The cell culture includes stomach epithelial cells, mucus, and an acidic pH. The parameters adjusted include a stomach permeability.

The platform 100 is configured to operate by circulating the fluid media through the target cells, enabling measurements of drug absorption to occur. In an example operation scenario, a mini-pill or API is put in input chamber 102 at a pH of about 1.5, incubated for a given amount of time (e.g., 5 minutes to an hour or more). An example input includes 500 μM at 100% solubility. After a period of time (e.g., 10-30 minutes), the solution from input 102 is be removed at a given volume, and the process is repeated (e.g., gastric emptying). The sample from input 102 is mixed with the solution from input 104 in the mixing compartment 105 to adjust the pH (e.g., measured by sensor 120) and to add bile and/or pancreatic enzymes. (e.g., mixing ˜100 μM). The mixed solution is injected from the mixing compartment into the cell chamber (e.g., a tissue culture compartment) including chambers 106, 108. These steps are repeated to mimic gastric emptying. The cell culture is recirculated after a number of hours to mimic transit times. The platform 100 collects samples in chambers 114, 116 from apical and basal media after a number of minutes.

A computing system (not shown) uses data developed from the platform 100 to combine the measurements of drug kinetics with data of the drug properties for training machine learning models in a processing workflow. Specifically, computational fluid dynamics (CFD) models are developed to guide design of the hardware circuit for the platform 100 in a first phase. The design parameters include compartment volumes, tissue size and/or surface area, flow path geometry, and media exchange and oxygenation chamber geometry and flow modeling. The operational parameters include gastric emptying time, a transit or recirculation period, a mixing time, and microbiota density and culture values. The hardware configuration of the platform 100 is tuned to a particular configuration (e.g., for a particular cell type).

The computational modeling is performed for target (e.g., drugs or drug combinations) kinetics modeling using the trained machine learning models and quantitative systems pharmacology (QSP) based models. These models are configured to identify how drugs or drug combinations are absorbed in tissues. The models link target composition data to kinetic data, described previously. For example, the computational model predicts concentration profiles based on membrane diffusion, metabolism, recirculation, and basal BSA binding. From these simulations, a data processing system can differentiate drugs using only a few samples for a wide range of permeabilities and metabolic clearances, with or without basal BSA.

FIG. 1B shows an example of a microphysiological system 115 that includes an apical chamber 108 and a basal chamber 106, similar to the microphysiological system 100 described in relation to FIG. 1A. The microphysiological system 115 includes a single pump 101. The pump 101 is configured to pump the fluid media through a single fluid loop 132. The fluid loop 132 includes the cell chamber 130 and a reoxygenation chamber 128. The cell chamber 130 includes a basal chamber 106 and an apical chamber 108, similar to the basal and apical chambers described in relation to FIG. 1A. A membrane 126, such as a semi permeable membrane, separates the apical chamber 108 and the basal chamber 106. The semi permeable membrane 126 is configured to allow fluid media to perfuse the opposing chamber. For example, the microphysiological system 115 has a single fluid loop 132 in which the pump 101 pumps the fluid media through the basal chamber 106. the fluid media can perfuse the cells in the apical chamber 108 through the semi permeable membrane 126.

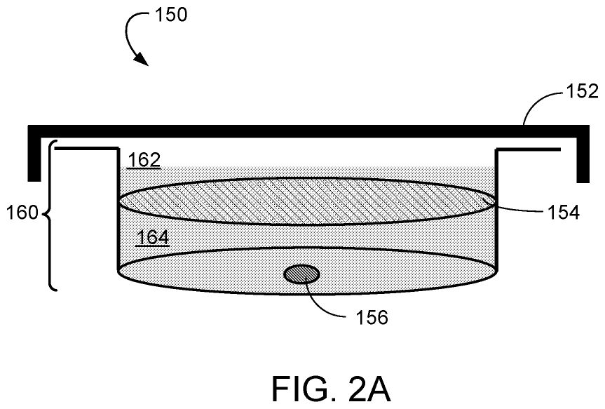

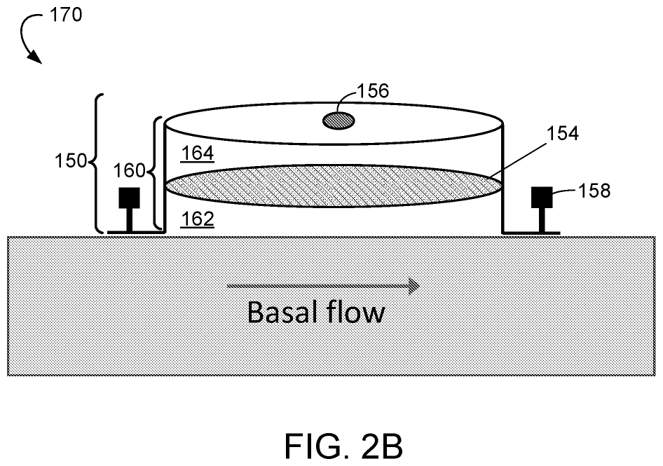

This cell chamber 130 can be a removable insert in which cells are cultured within either the apical chamber 108 or the basal chamber 106 prior to insertion of the cell chamber 130 into the fluid loop 132. The removable insert including the cell chamber 130 is described in more detail in relation to FIGS. 2A-2B. As subsequently described, the cell chamber 130 is an insert that enables the user to switch between the apical chamber 108 and the basal chamber 106 as being in line with the fluid loop 132. The pump 101 is configured to pump fluid media through the re oxygenation chamber 128 and through the basal chamber 106, as shown in FIG. 1B, or the apical chamber 108, depending on how the user has inserted the cell chamber 130 into the fluid loop 132. The reoxygenation chamber 128 can be similar to the reoxygenation chamber 110 or the reoxygenation chamber 112 depending on whether the basal chamber 106 or the apical chamber 108 of the cell chamber 130 is in the fluid loop 132.

FIG. 1C shows an example of a microphysiological system 117 that includes two fluid loops. Microphysiological system 117 can be similar to the microphysiological system 100, described in relation to FIG. 1A. A first fluid loop 132 includes a pump 103 configured to pump fluid media through the basal chamber 106 and the reoxygenation chamber 110. A second fluid loop 134 includes a pump 107 configured to pump fluid media through the apical chamber 108 and the reoxygenation chamber 112. In the example of the microphysiological system 117, the two fluid loops 132, 134 are operable in parallel with two different pumps 103, 107, respectively. In this configuration, the cell chamber 130 is inserted into the microphysiological system 117 in a particular orientation such that the pump 107 pumps fluid media through the apical chamber 108 and the pump 103 pumps fluid media through the basal chamber 106. The cell chamber 130 can be removed for seeding the cells in each of the chambers 106, 108 prior to inserting the cell chamber 130 into yeah microphysiological system 117 to interface with each of the fluid loops 132, 134. In some implementations, the pumps 103, 107 can be separately controlled by a controller. For example, pump 103 can pump fluid media in fluid loop 132 at a first rate, and pump 107 can pump fluid media through fluid loop 134 at a second, different rate. Fluid media can be pumped in parallel through the apical chamber 108 at a different rate than fluid media are pumped through the basal chamber 106. In some implementations, the cell chamber 130 is an insert that can be flipped such that the basal chamber 106 is inserted into fluid loop 134, and apical chamber 108 is inserted into fluid loop 132. As previously described, the cells in each of the chambers 106, 108 can be seeded in advance of perfusion of the fluid media through their respective fluid loops 132, 134.

FIG. 1D shows an example of a microphysiological system 125 that includes three different pumps. Microphysiological system 125 is similar to the microphysiological system 100 described in relation to FIG. 1A except that there are three pumps included instead of two pumps. A first pump 109 pumps fluid media from a drug mixing or addition module 105. A second pump 111 pumps fluid media through an apical fluid loop 134. A third pump 113 pumps food media through a basal fluid loop 132. As previously described, the cell chamber 130 includes an ethical chamber 108, a basal chamber 106, and semi permeable membrane 126 dividing the basal chamber from the apical chamber. The pumps 111 and 113 can pump fluid media at different rates through each of the apical chamber 108 and the basal chamber 106, respectively. The pump 109 can pump at a different rate than either pump 111 or pump 113. In microphysiological system 125, the drug mixing or addition module 105 introduces drugs directly into the apical fluid loop 134. In some implementations, the drug mixing or addition module 105 can introduce a drug mixture into the basal fluid loop 132.

FIG. 2A shows an example insert 150 that includes the cell chamber 160, which can be similar to cell chamber 130 of FIGS. 1A-1D. The insert 150 is configured to be inserted into one or more of the microphysiological systems 100, 115, 117, and 125, previously described. The insert 150 is configured to be seated in an upside down configuration, as shown in FIG. 2A. A cover 152 is configured to cover a cell chamber 160 that includes two sub-chambers 162, and 164. A first chamber 162 can be similar to the basal chamber 106, previously described. The second chamber 164 can be similar to the apical chamber 108, previously described. The second chamber 164 can include a seeding port 156. The first chamber 162 and the second chamber 164 can be separated by a membrane 154, such as a semi permeable membrane it was described previously. The seeding port 156 can be used to introduce cells into the chamber 164 on a second apical) side of the membrane 154. The sales can be added to the sale chamber 160 prior to inserting insert 150 into the microphysiological devices described herein. In an example, the cells can be added to the cell insert 150 and later perfused with fluid media after the insert is added to a microphysiological system.

FIG. 2B shows an example of the insert 150 after seeding has occurred and the insert has been added to a microphysiological system 170, such as systems 100, 115, 117, 125. coupling devices 158 such as thumb screws, can passing the insert 150 to the fluid loop of the microphysiological device 170. In this example, the insert 150 is fastened to the basal fluid loop such that chamber 162 is a basal fluid chamber. The cells can be in chamber 164 on the other side of the membrane 154. In some implementations, the insert 150 can be monolithic.

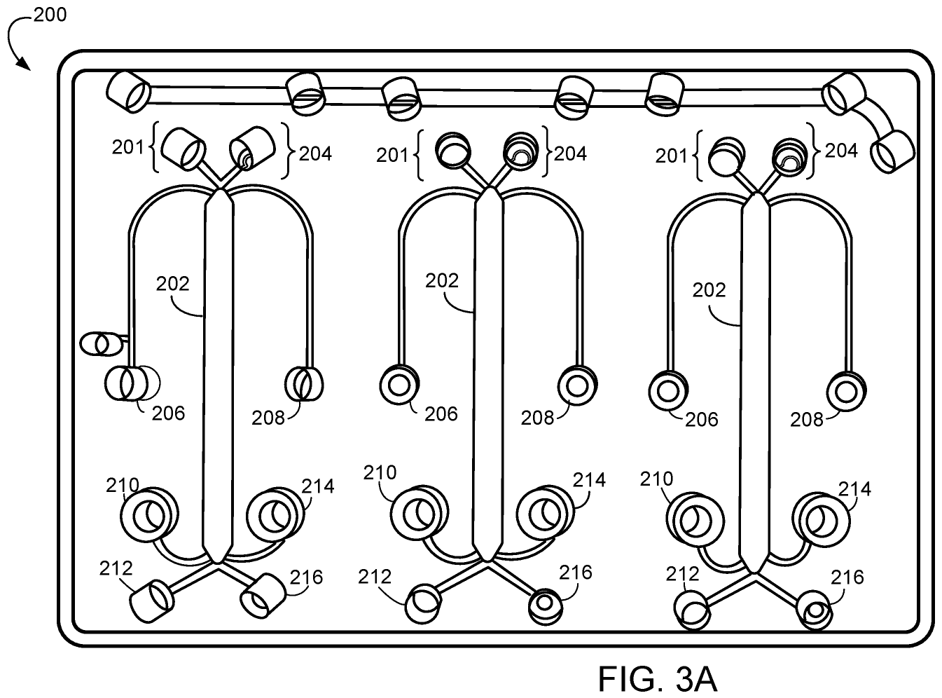

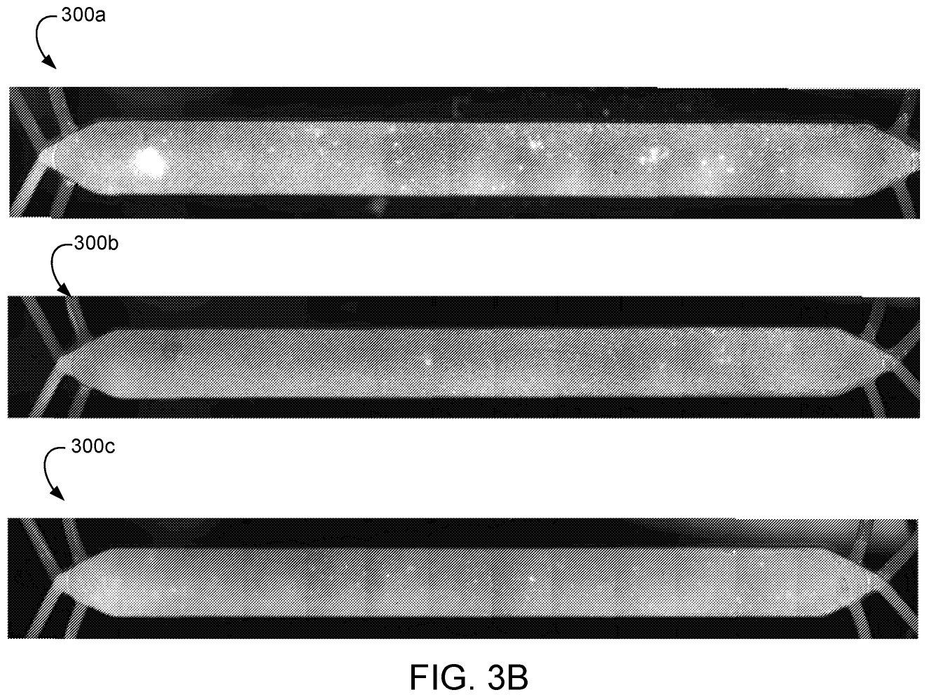

FIG. 3A is an image showing a top view of an MPS layer 200 including cell culture chambers 202 and associated ports for the microfluidic platform for drug absorption analysis of FIG. 1. For example, the chambers 202 each represent an instance of a two compartment chamber including the apical chamber 108 and the basal chamber 106 of FIGS. 1A-1D. Because the top view is shown for layer 200, only the apical chamber 108 is visible in the image. Each of the basal chamber 106 and the apical chamber 108 of the cell culture chamber 202 are associated with an apical perfusion port (inlet) 201, a basal perfusion port (inlet) 204, an apical perfusion port (outlet) 212, a basal perfusion port (outlet) 216, an apical seeding port (inlet) 206, a basal seeding port (inlet) 208, an apical seeding port (outlet) 210, and a basal seeding port (outlet) 214. In FIG. 3A, each of the cell culture chambers 202 is shown with gut cells that form a confluent monolayer in the cell culture chamber 202.

FIG. 3B includes images of the cell cultures 300a-c in the cell chambers of FIG. 3A. For example, these cell cultures 300a-c each include P13 Caco-2 Bbe1 cells on day 9 of culturing with a collagen I-coated two compartment chamber (e.g., apical and basal compartments). As shown in each of cell cultures 300a-c, the tissues can be cultured on the apical membrane.

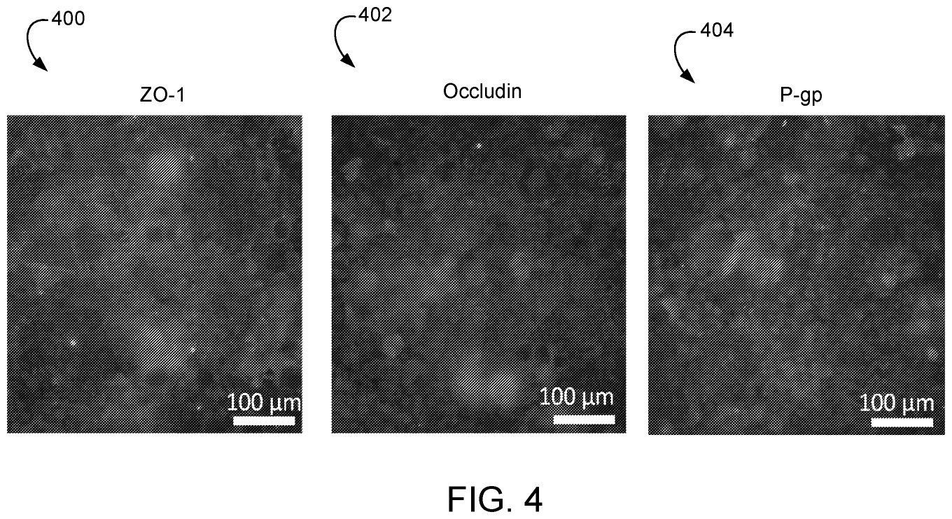

FIG. 4 shows an example of cell cultures from FIGS. 3A-3B, in which the gut cells are stained for junction proteins and transporters. For example, image 400 shows stained ZO-1 cells at 100 um. Image 402 shows an example of Occludin at 100 um. Image 404 shows P-gp cells at 100 um.

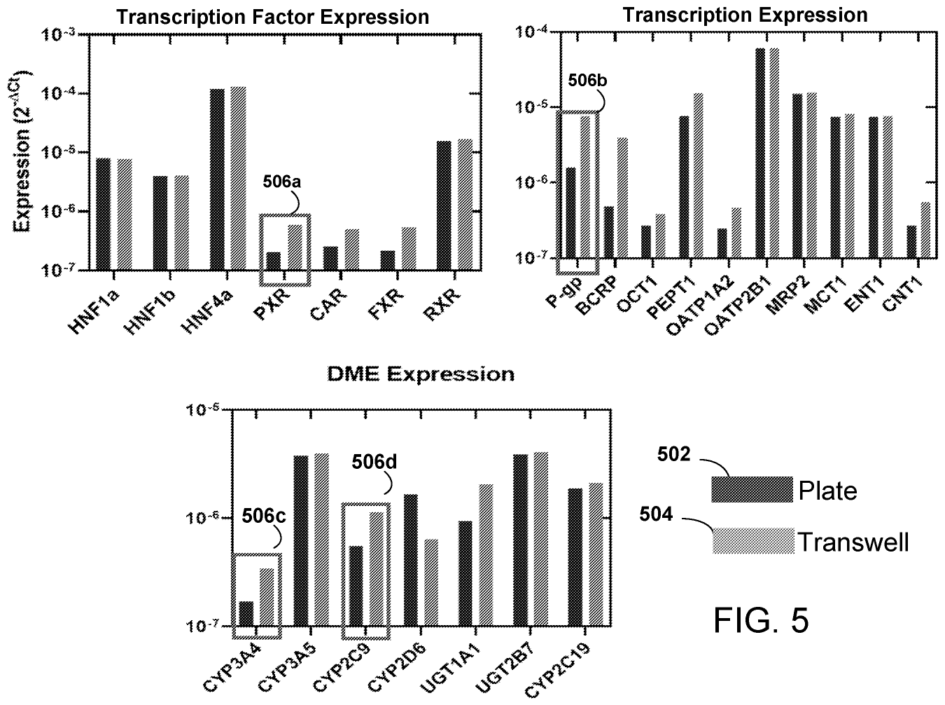

FIG. 5 shows graphs 500 including a comparison of Caco-2 cells under different culture conditions from static cultures. These include Caco-2 Bbel cells cultured on collagen I-coated plates and transwells at day 23, 18S endogenous control. The plate 502 cultures and transwell 604 cultures are compared (e.g., shown in pairs in example pairs in boxes 506a-d). Each box 506a-d shows a differential effect on a respective given gene. The transwell conditions include the tissue culture on a membrane with apical and basal media. The plate conditions include tissue on a plate with single compartment. As shown in FIG. 5, transwell conditions generally improve PK-related gene expression in gut culture.

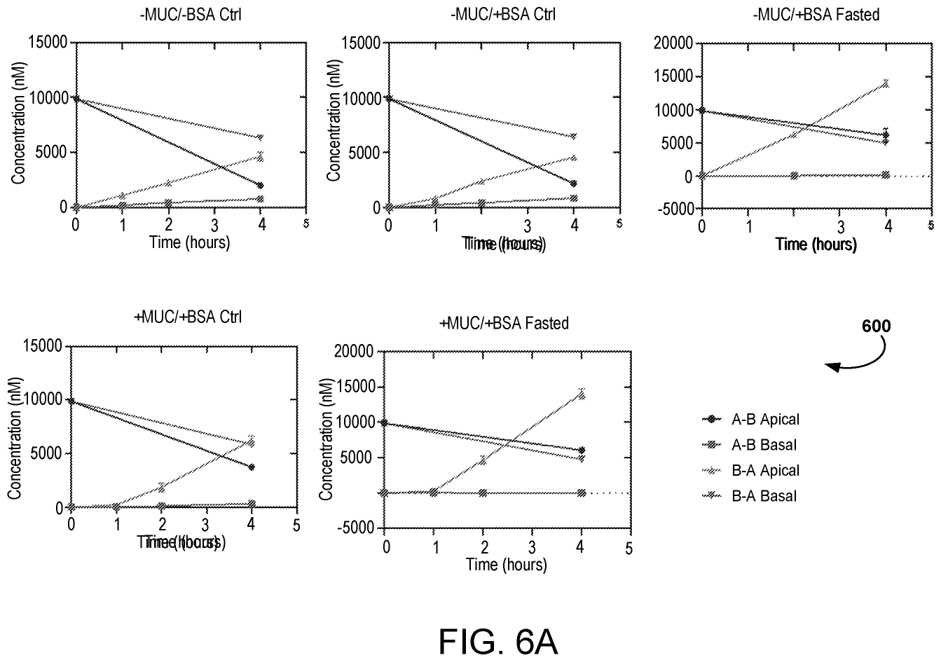

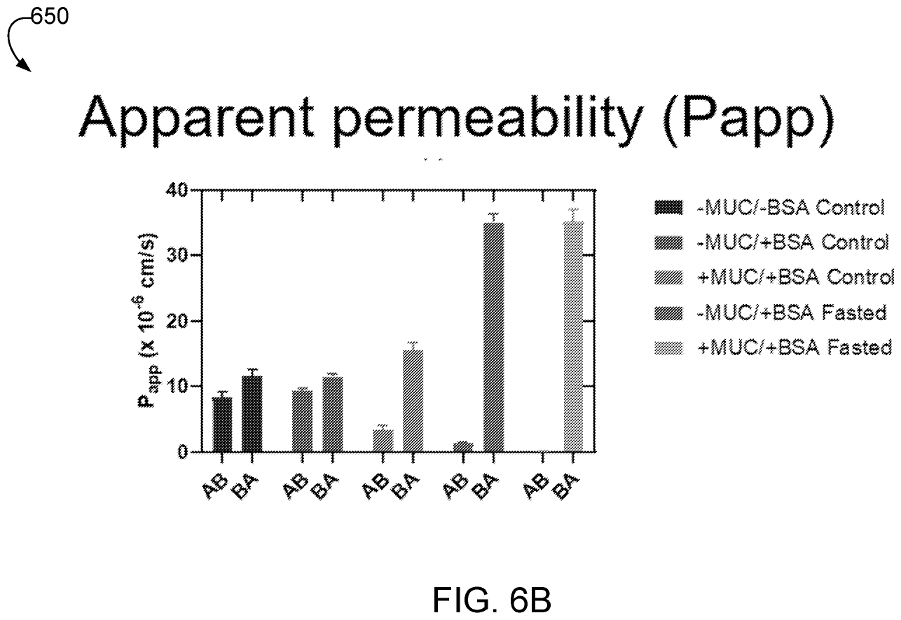

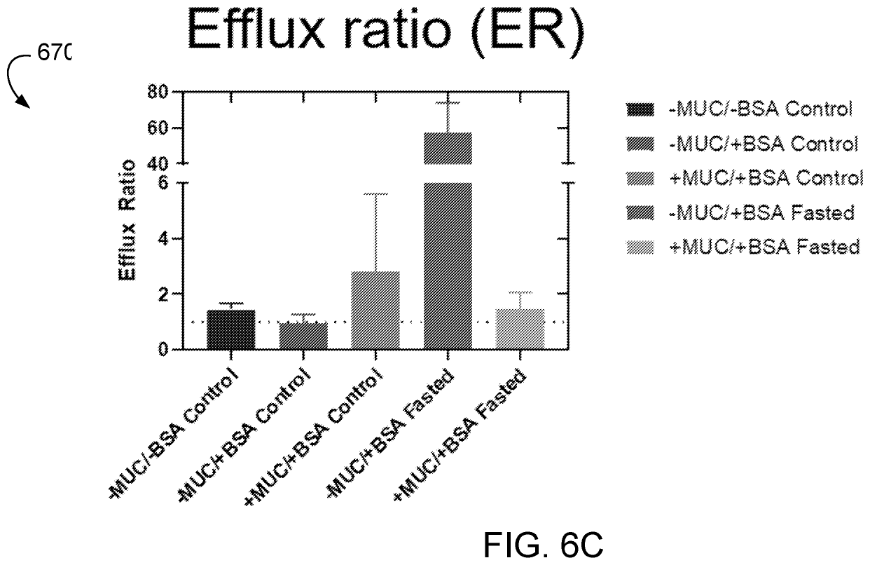

FIGS. 6A-6C show example data of measured effects of luminal mucin, basal BSA & fasted state on propranolol permeability on static Caco-2 cultures. The results 600, 650, and 670 show that the gut tissue microenvironment (e.g., mucin & pH) affects drug transport. The microenvironment is maintained using the MPS 100 described in relation to FIG. 1. For example, gastric emptying changes a pH on the culture (e.g., flow from mixing module 105).

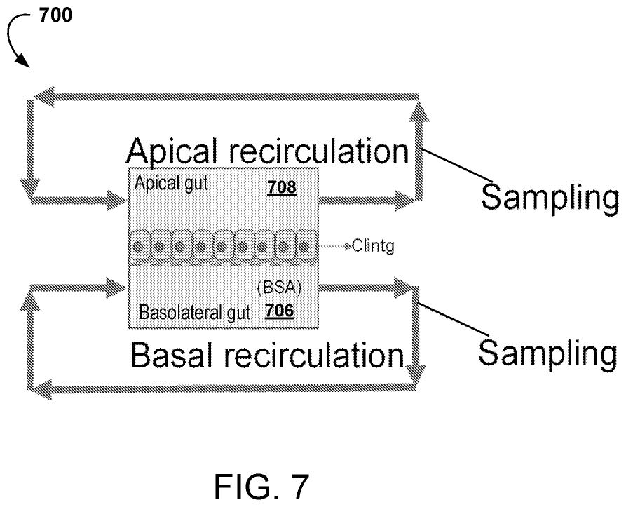

FIG. 7 shows a diagram of an example cell culture chamber 700 including an apical sub-chamber 708 and a basal sub-chamber 706. In some implementations, the chamber 700 is similar to chambers 200 of FIG. 3A. The apical sub-chamber is similar to apical chamber 108 of FIG. 1. The basolateral gut chamber 106 is similar to chamber 108 of FIG. 1. A computational model is configured to predict concentration profiles with membrane diffusion, metabolism, recirculation, and basal BSA binding for the chamber 700. Example design parameters can include the following. MPS design parameters include an apical flow rate, a basal flow rate, and a gut membrane area. In this example, the apical flow rate is about 10 mL/day. In this example, the basal flow rate is about 10 mL/day. In this example, the gut membrane area is 1cm². The example design parameters can include compartment volumes. In this example, the apical compartment volume is 0.2 mL. In this example, the apical recirculation is 1 mL. In this example, the basal compartment is 0.2 mL. In this example, the basal recirculation is 3 mL. The example design parameters can include drug-related parameters. For example, the drug dose is 10 uM. In this example, the BSA binding is one of 0%, 50%, or 90%. In this example, the gut metabolism is 0, 0.1 mL/hour, or 1 mL/hour. In this example, the permeability is 1×10⁻⁶ cm/s, 10×10⁻⁶ cm/s, or 100×10⁻⁶ cm/s. Other values for the design parameters are possible in addition to these example values.

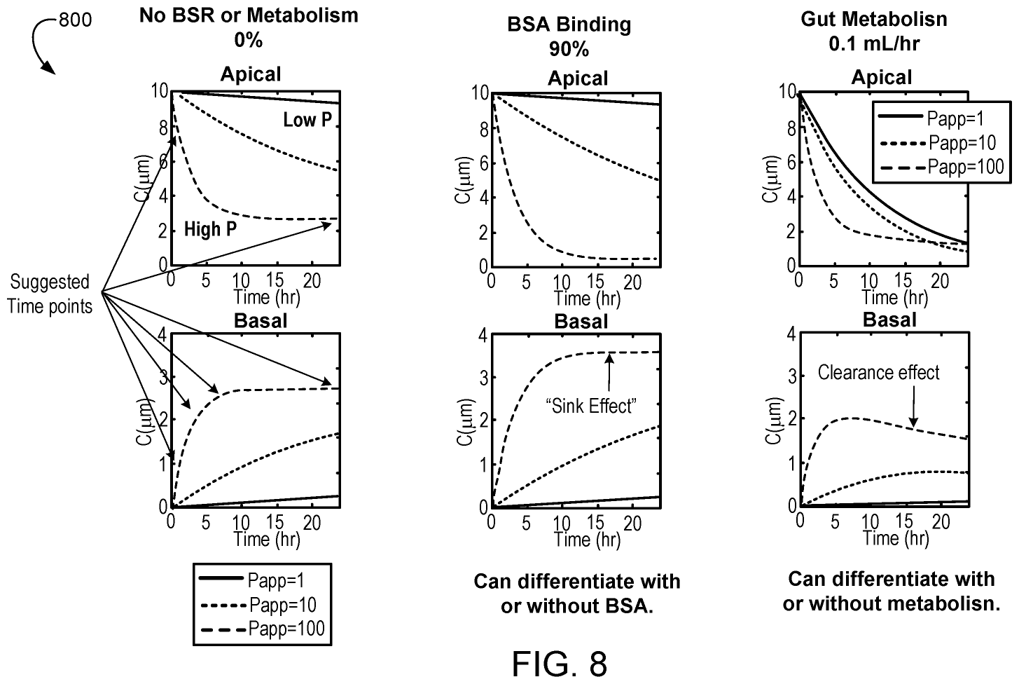

FIG. 8 shows example results data 800 from computational simulations for determining chip design and operational parameters including flow rate and media composition. Example time points for sampling the fluid media occur at 1 hour and 24 hours for apical recirculation. Example time points for sampling the fluid media occur at 1 hour, 4 hours, 8 hours, and 24 hours for basal recirculation. These simulated data show that drugs can be differentiated using a small number of samples for a wide range of wide range of permeabilities and metabolic clearances with or without basal BSA.

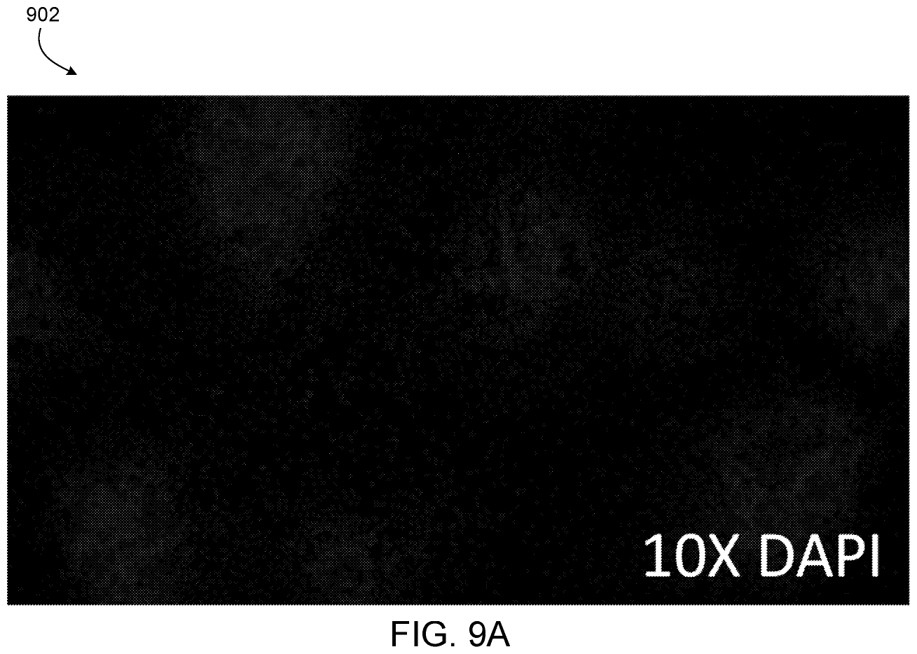

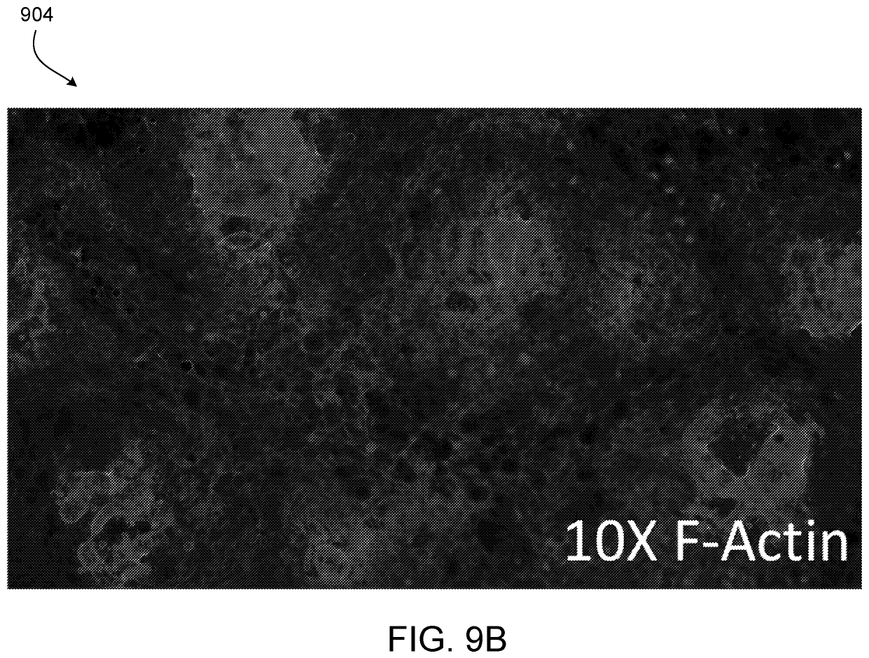

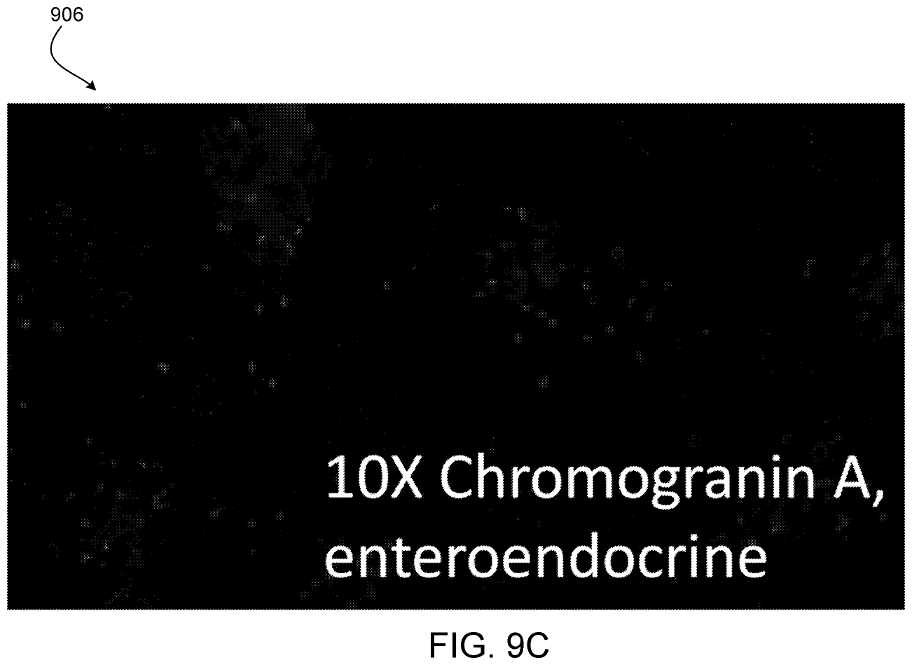

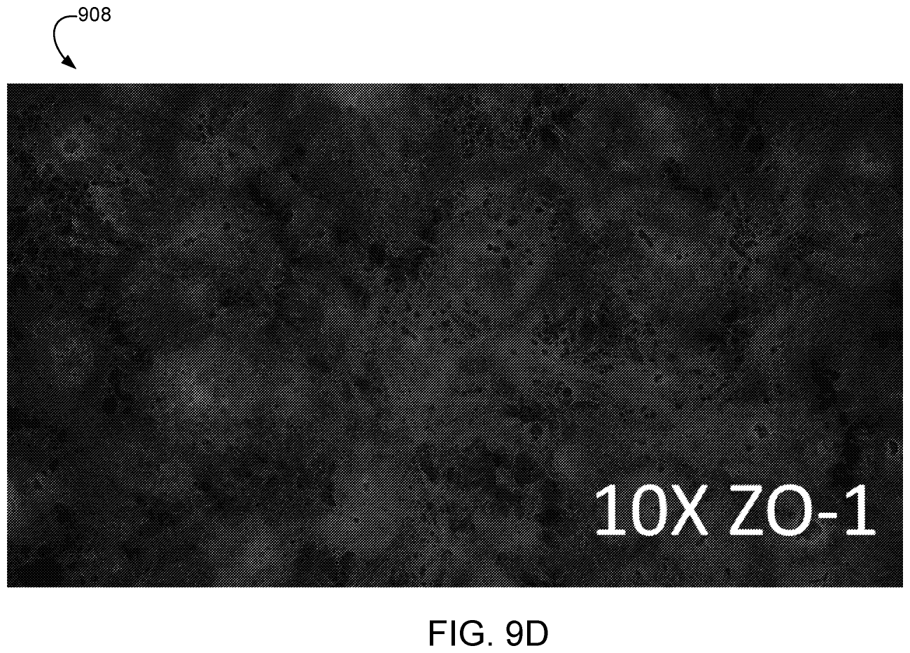

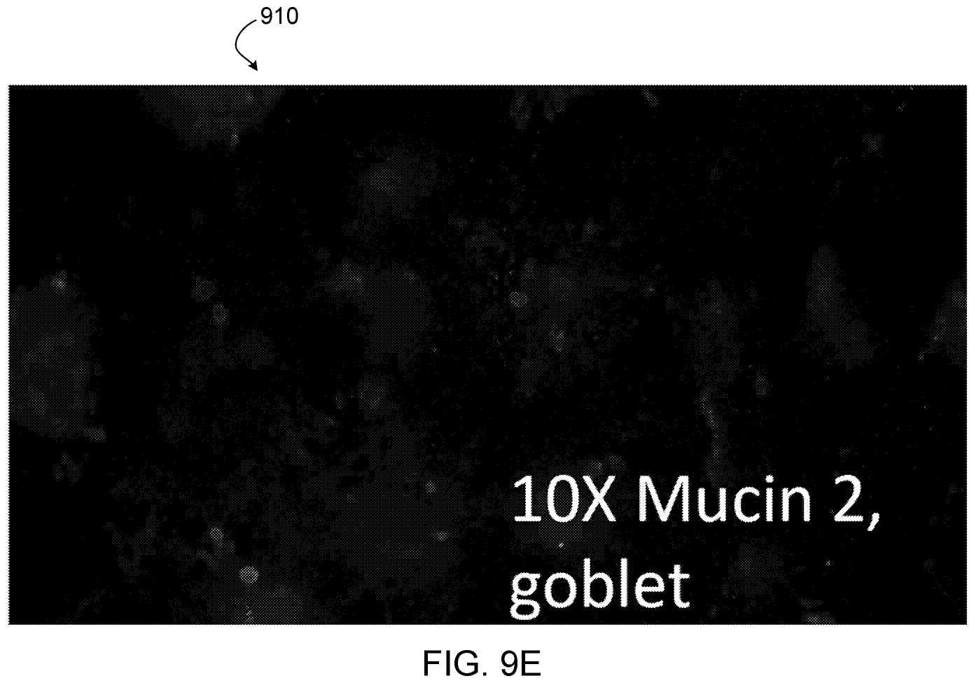

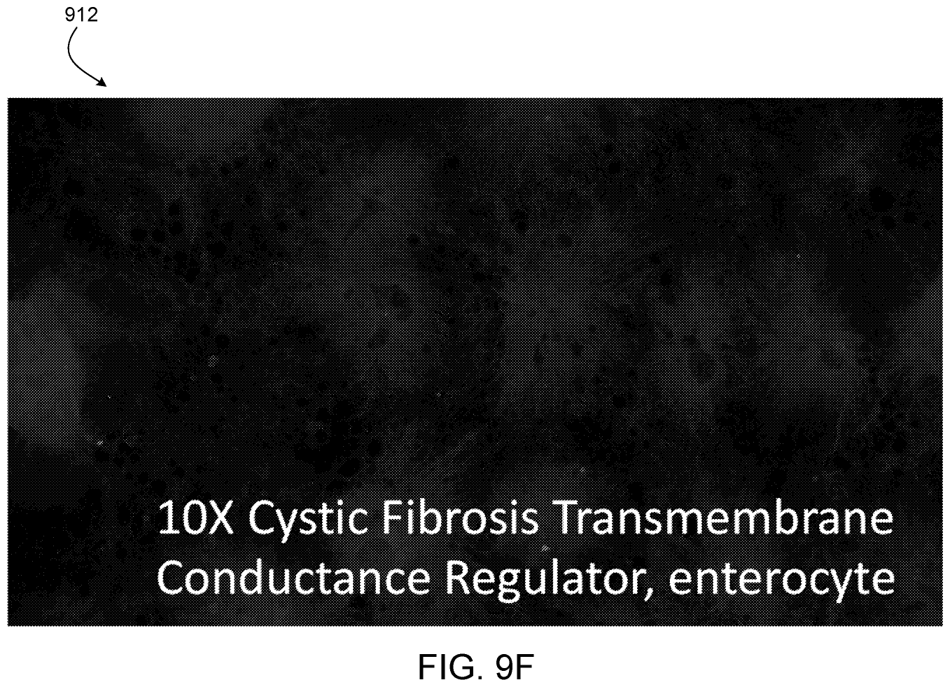

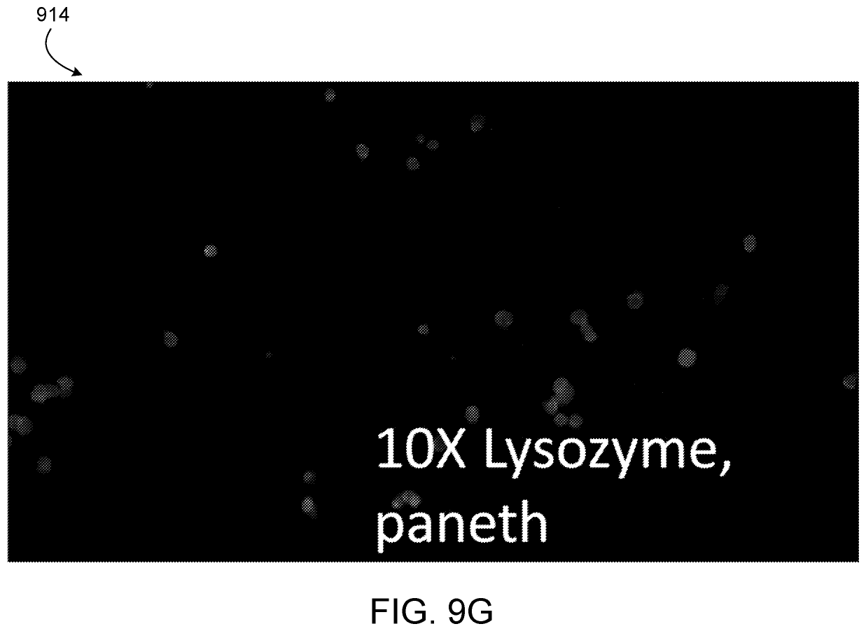

FIGS. 9A-9G show examples of cell cultures that are cultured in the apical chamber or the basal chamber of the MPS 100 of FIG. 1. FIG. 9A shows a 10× 4′, 6-diamidino-2-phenylindole (DAPI) cell culture 902. FIG. 9B shows a 10× F-Actin cell culture 904. FIG. 9C shows a 10× Chromogranin A, enteroendocrine cell culture 906. FIG. 9D shows a 10× ZO-1 cell culture 908. FIG. 9E shows a 10× Mucin 2, goblet cell culture 910. FIG. 9F shows a 10× Cystic Fibrosis transmembrane conductance regulator, enterocyte cell culture 912. FIG. 9G shows a 10× lysozyme paneth cell culture 914. Each cell culture 902, 904, 906, 908, 910, 912, and 914 can be cultured in either the apical cell chamber 108 or the basal cell chamber 106. Each of the cell culture examples 902, 904, 906, 908, 910, 912, and 914 are cultured under perfusion using the chamber 200.

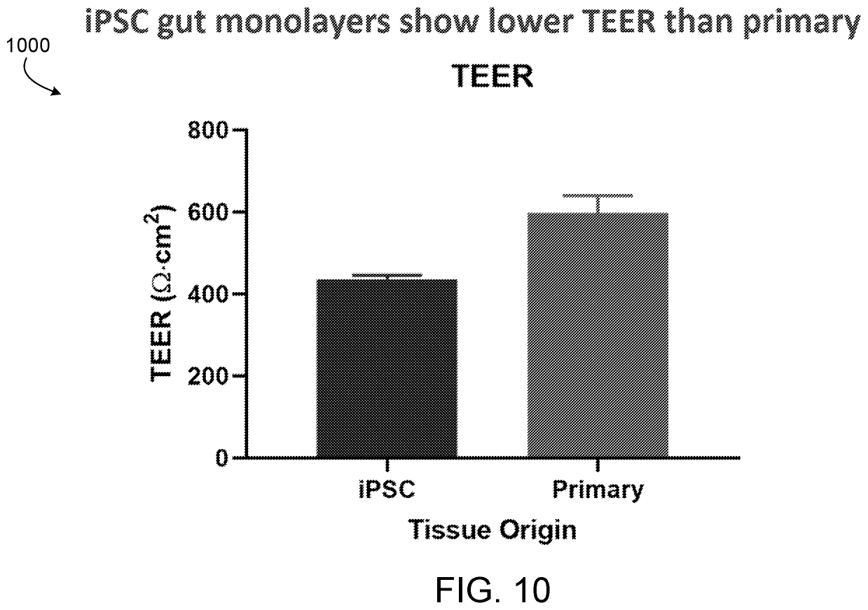

FIG. 10 is a graph 1000 showing TEER values (in Ω·cm2) for different tissue origins. The tissue origins include iPSC and primary cell cultures. Here, iPSC gut monolayers have lower TEER levels than primary cell cultures. Both TEER values (>250) indicate barrier function of the tissue (e.g., tight junctions). A lower value of iPSC does not have any negative implication.

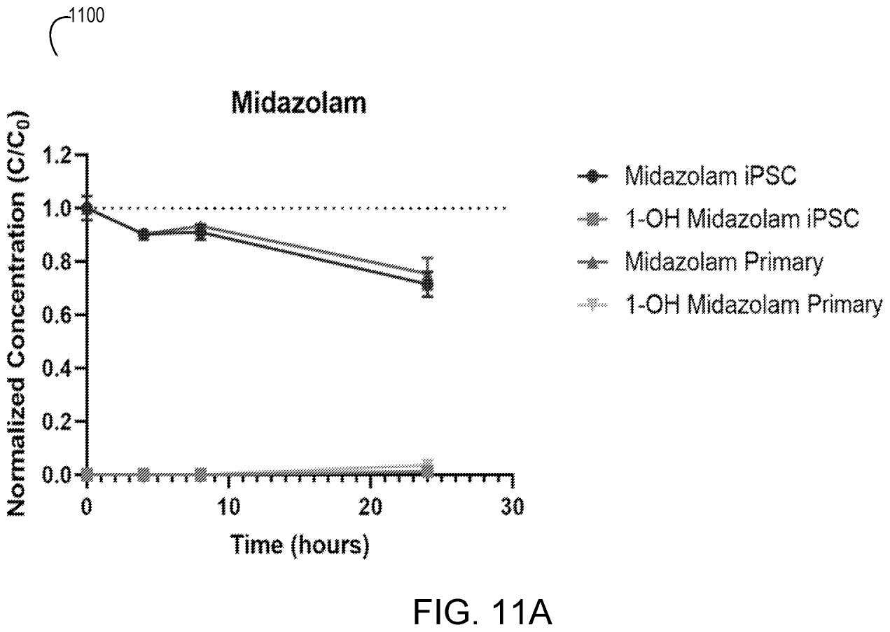

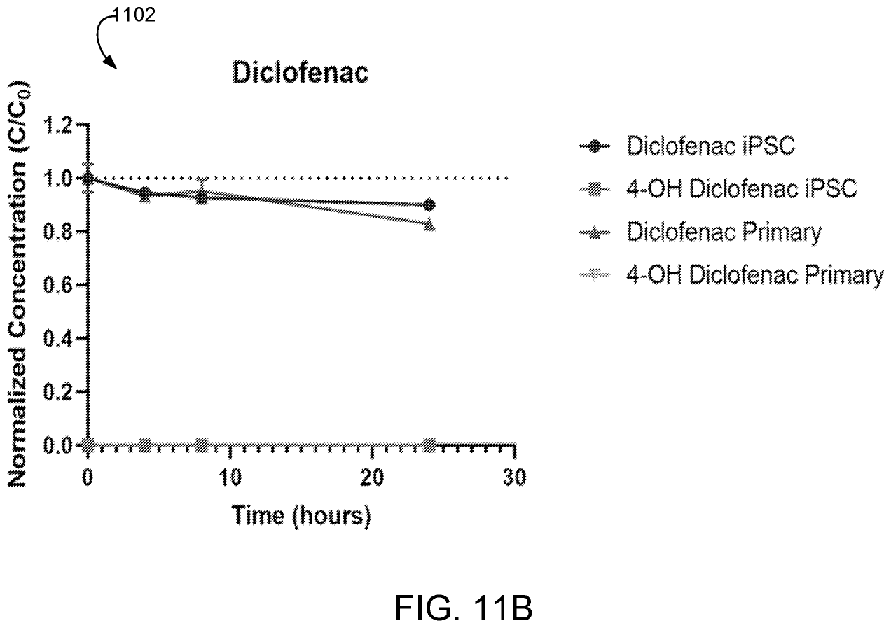

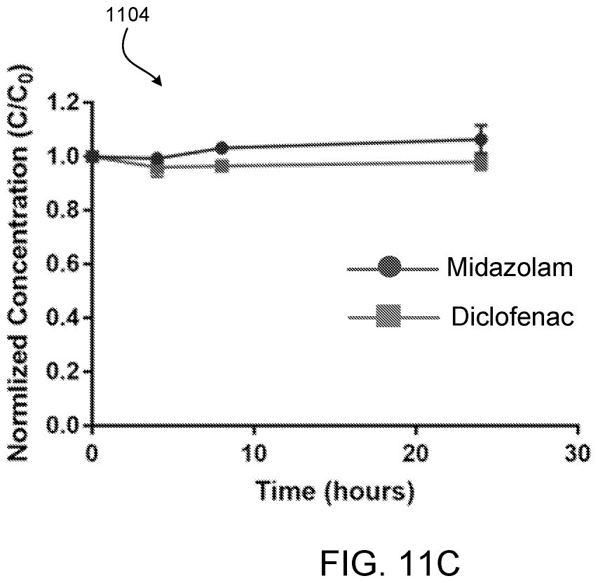

FIGS. 11A-11C show graphs indicating concentration values over time for different iPSC tissue. Graph 1100 of FIG. 11A shows a normalized concentration (C/C₀) for Midazolam over time (hours). Each of Midazolam iPSC, 1-OH Midazolam iPSC, Midazolam primary, and 1-OH Midazolam primary are shown. Graph 1100 shows a normalized concentration (C/C₀) for Midazolam over time (hours). Each of Midazolam iPSC, 1-OH Midazolam iPSC, Midazolam primary, and 1-OH Midazolam primary are shown. Graph 1102 of FIG. 11B shows a normalized concentration (C/Co) for Diclofenac over time (hours). Each of Diclofenac iPSC, 4-OH Diclofenac iPSC, Diclofenac primary, and 4-OH Diclofenac primary are shown. Graph 1104 of FIG. 11C shows a normalized concentration (C/Co) for Diclofenac and Midazolam over time (hours).

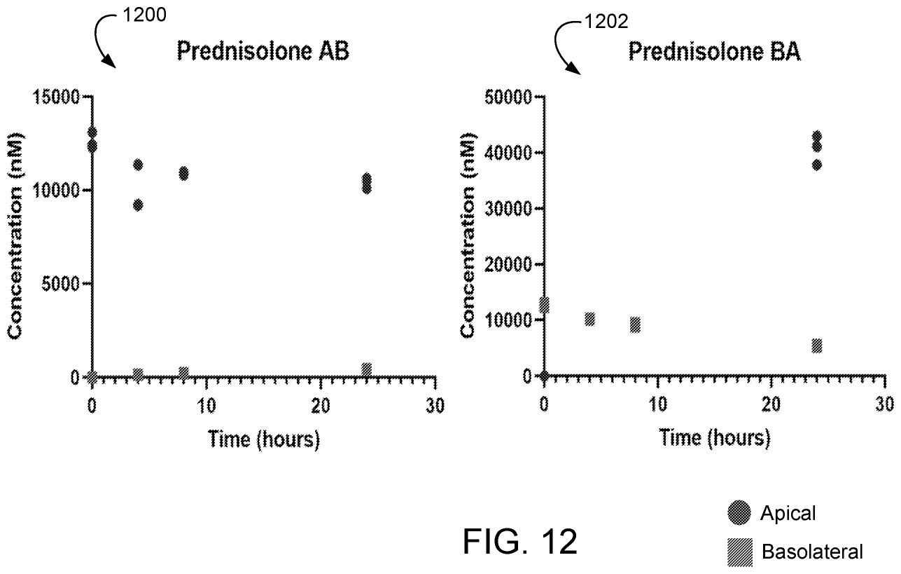

FIG. 12 show graphs 1200, 1202 indicating concentration values over time for iPSC-derived gut tissue, including apical-to-basal and basal-to-apical concentrations for prednisolone using for iPSC-derived gut tissue. Graph 1200 shows concentrations over time for prednisolone apical-to-basal (AB). Graph 1202 shows concentrations for Prednisolone (basal-to-apical).

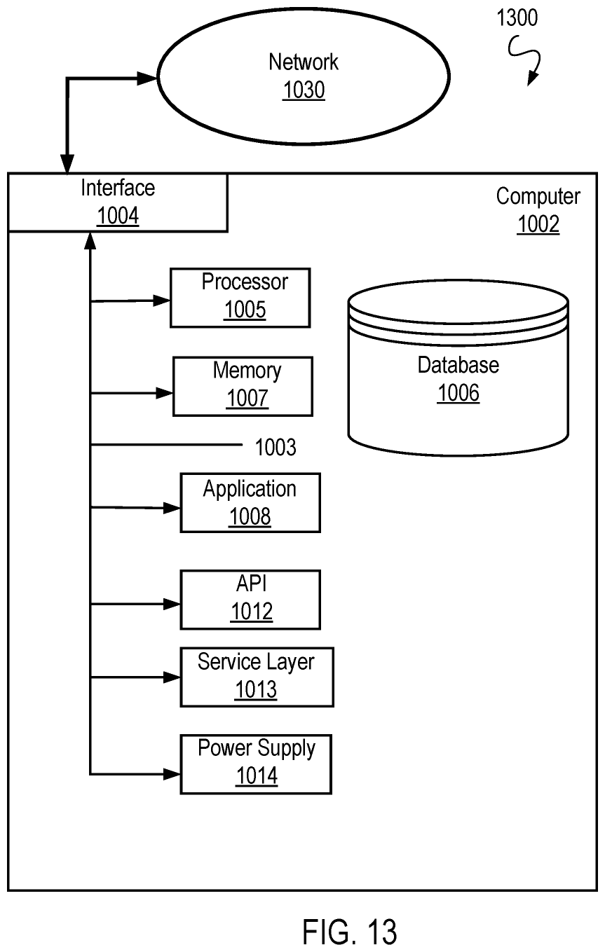

FIG. 13 is a block diagram of an example computer system 1300 used to provide computational functionalities associated with described algorithms, methods, functions, processes, flows, and procedures described in the present disclosure according to some implementations of the present disclosure. The illustrated computer 1002 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 1002 can include input devices such as keypads, keyboards, and touch screens that can accept user information. Also, the computer 1002 can include output devices that can convey information associated with the operation of the computer 1002. The information can include digital data, visual data, audio information, or a combination of information. The information can be presented in a graphical user interface (UI or GUI).

The computer 1002 can serve in a role as a client, a network component, a server, a database, a persistency, or components of a computer system for performing the subject matter described in the present disclosure. The illustrated computer 1002 is communicably coupled with a network 1030. In some implementations, one or more components of the computer 1002 can be configured to operate within different environments, including cloud-computing-based environments, local environments, global environments, and combinations of environments.

At a high level, the computer 1002 is an electronic computing device operable to receive, transmit, process, store, and manage data and information associated with the described subject matter. According to some implementations, the computer 1002 can also include, or be communicably coupled with, an application server, an email server, a web server, a caching server, a streaming data server, or a combination of servers.

The computer 1002 can receive requests over network 1030 from a client application (for example, executing on another computer 1002). The computer 1002 can respond to the received requests by processing the received requests using software applications. Requests can also be sent to the computer 1002 from internal users (for example, from a command console), external (or third) parties, automated applications, entities, individuals, systems, and computers.

Each of the components of the computer 1002 can communicate using a system bus 1003. In some implementations, any or all of the components of the computer 1002, including hardware or software components, can interface with each other or the interface 1004 (or a combination of both), over the system bus 1003. Interfaces can use an application programming interface (API) 1012, a service layer 1013, or a combination of the API 1012 and service layer 1013. The API 1012 can include specifications for routines, data structures, and object classes. The API 1012 can be either computer-language independent or dependent. The API 1012 can refer to a complete interface, a single function, or a set of APIs.

The service layer 1013 can provide software services to the computer 1002 and other components (whether illustrated or not) that are communicably coupled to the computer 1002. The functionality of the computer 1002 can be accessible for all service consumers using this service layer. Software services, such as those provided by the service layer 1013, can provide reusable, defined functionalities through a defined interface. For example, the interface can be software written in JAVA, C++, or a language providing data in extensible markup language (XML) format. While illustrated as an integrated component of the computer 1002, in alternative implementations, the API 1012 or the service layer 1013 can be stand-alone components in relation to other components of the computer 1002 and other components communicably coupled to the computer 1002. Moreover, any or all parts of the API 1012 or the service layer 1013 can be implemented as child or sub-modules of another software module, enterprise application, or hardware module without departing from the scope of the present disclosure.

The computer 1002 includes an interface 1004. Although illustrated as a single interface 1004 in FIG. 10, two or more interfaces 1004 can be used according to particular needs, desires, or particular implementations of the computer 1002 and the described functionality. The interface 1004 can be used by the computer 1002 for communicating with other systems that are connected to the network 1030 (whether illustrated or not) in a distributed environment. Generally, the interface 1004 can include, or be implemented using, logic encoded in software or hardware (or a combination of software and hardware) operable to communicate with the network 1030. More specifically, the interface 1004 can include software supporting one or more communication protocols associated with communications. As such, the network 1030 or the interface's hardware can be operable to communicate physical signals within and outside of the illustrated computer 1002.

The computer 1002 includes a processor 1005. Although illustrated as a single processor 1005 in FIG. 10, two or more processors 1005 can be used according to particular needs, desires, or particular implementations of the computer 1002 and the described functionality. Generally, the processor 1005 can execute instructions and can manipulate data to perform the operations of the computer 1002, including operations using algorithms, methods, functions, processes, flows, and procedures as described in the present disclosure.

The computer 1002 also includes a database 1006 that can hold data for the computer 1002 and other components connected to the network 1030 (whether illustrated or not). For example, database 1006 can be an in-memory, conventional, or a database storing data consistent with the present disclosure. In some implementations, database 1006 can be a combination of two or more different database types (for example, hybrid in-memory and conventional databases) according to particular needs, desires, or particular implementations of the computer 1002 and the described functionality. Although illustrated as a single database 1006 in FIG. 10, two or more databases (of the same, different, or combination of types) can be used according to particular needs, desires, or particular implementations of the computer 1002 and the described functionality. While database 1006 is illustrated as an internal component of the computer 1002, in alternative implementations, database 1006 can be external to the computer 1002.

The computer 1002 also includes a memory 1007 that can hold data for the computer 1002 or a combination of components connected to the network 1030 (whether illustrated or not). Memory 1007 can store any data consistent with the present disclosure. In some implementations, memory 1007 can be a combination of two or more different types of memory (for example, a combination of semiconductor and magnetic storage) according to particular needs, desires, or particular implementations of the computer 1002 and the described functionality. Although illustrated as a single memory 1007 in FIG. 10, two or more memories 1007 (of the same, different, or combination of types) can be used according to particular needs, desires, or particular implementations of the computer 1002 and the described functionality. While memory 1007 is illustrated as an internal component of the computer 1002, in alternative implementations, memory 1007 can be external to the computer 1002.

The application 1008 can be an algorithmic software engine providing functionality according to particular needs, desires, or particular implementations of the computer 1002 and the described functionality. For example, application 1008 can serve as one or more components, modules, or applications. Further, although illustrated as a single application 1008, the application 1008 can be implemented as multiple applications 1008 on the computer 1002. In addition, although illustrated as internal to the computer 1002, in alternative implementations, the application 1008 can be external to the computer 1002.

The computer 1002 can also include a power supply 1014. The power supply 1014 can include a rechargeable or non-rechargeable battery that can be configured to be either user-or non-user-replaceable. In some implementations, the power supply 1014 can include power-conversion and management circuits, including recharging, standby, and power management functionalities. In some implementations, the power-supply 1014 can include a power plug to allow the computer 1002 to be plugged into a wall socket or a power source to, for example, power the computer 1002 or recharge a rechargeable battery.

There can be any number of computers 1002 associated with, or external to, a computer system containing computer 1002, with each computer 1002 communicating over network 1030. Further, the terms “client,” “user,” and other appropriate terminology can be used interchangeably, as appropriate, without departing from the scope of the present disclosure. Moreover, the present disclosure contemplates that many users can use one computer 1002 and one user can use multiple computers 1002.

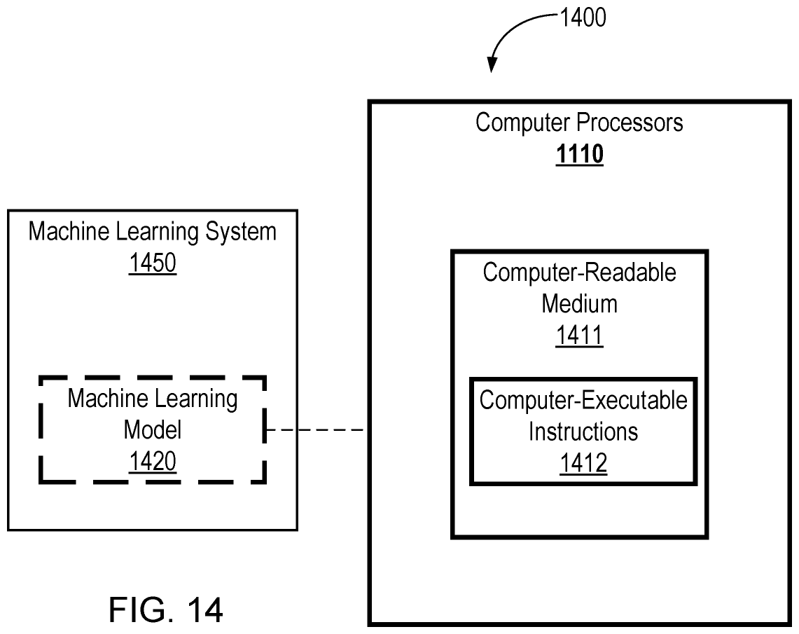

FIG. 14 is a diagram illustrating an example computer system 1400 configured to execute a machine learning model. Generally, the computer system 1400 is configured to process data indicating a phenotype and determine a class label (“healthy” or “disease”) to predict the effect of a target on the state of the MPS. The system 1400 includes computer processors 1410. The computer processors 1410 include computer-readable memory 1411 and computer readable instructions 1412. The system 1400 also includes a machine learning system 1450. The machine learning system 1450 includes a machine learning model 1420. The machine learning model 1420 can be separate from or integrated with the computer processors 1410.

The computer-readable medium 1411 (or computer-readable memory) can include any data storage technology type which is suitable to the local technical environment, including but not limited to semiconductor based memory devices, magnetic memory devices and systems, optical memory devices and systems, fixed memory, removable memory, disc memory, flash memory, dynamic random-access memory (DRAM), static random-access memory (SRAM), electronically erasable programmable read-only memory (EEPROM) and the like. In an embodiment, the computer-readable medium 1411 includes code-segment having executable instructions.

In some implementations, the computer processors 1410 include a general purpose processor. In some implementations, the computer processors 1410 include a central processing unit (CPU). In some implementations, the computer processors 1410 include at least one application specific integrated circuit (ASIC). The computer processors 1410 can also include general purpose programmable microprocessors, graphic processing units, special-purpose programmable microprocessors, digital signal processors (DSPs), programmable logic arrays (PLAs), field programmable gate arrays (FPGA), special purpose electronic circuits, etc., or a combination thereof. The computer processors 1410 are configured to execute program code means such as the computer-executable instructions 1412 and configured to execute executable logic that includes the machine learning model 1420.

The computer processors 1410 are configured to receive data indicating a molecular structure of, for example, a drug. The data can be obtained through one or more means, such as wireless communications with databases, optical fiber communications, USB, CD-ROM, and so forth.

The machine learning model 1420 is capable of processing the data to determine the class of phenotype (“healthy or disease”). In some implementations, the machine learning model 1420 is trained to determine class using a data set that includes MPS data (e.g., phenotypic, transcriptomic, etc.) and MPS labels. The machine learning model 1420 can classify the phenotype predicted by in vitro or in silico target perturbations. Accordingly, when a data set (in vitro or in silico) is introduced to the machine learning model 1420, it can predict whether an MPS platform exhibits a healthy or disease phenotype.

The machine learning system 1450 is capable of applying machine learning techniques to train the machine learning model 1420. As part of the training of the machine learning model 1420, the machine learning system 1450 forms a training set of input data by identifying a positive training set of input data items that have been determined to have the property in question, and, in some embodiments, forms a negative training set of input data items that lack the property in question.

The machine learning system 1450 extracts feature values from the input data of the training set, the features being variables deemed potentially relevant to whether or not the input data items have the associated property or properties. An ordered list of the features for the input data is herein referred to as the feature vector for the input data. In one embodiment, the machine learning system 1450 applies dimensionality reduction (e.g., via linear discriminant analysis (LDA), principle component analysis (PCA), or the like) to reduce the amount of data in the feature vectors for the input data to a smaller, more representative set of data.

In some implementations, the machine learning system 1450 uses supervised machine learning to train the machine learning models 1420 with the feature vectors of the positive training set and the negative training set serving as the inputs. Different machine learning techniques-such as linear support vector machine (linear SVM), boosting for other algorithms (e.g., AdaBoost), neural networks, logistic regression, naïve Bayes, memory-based learning, random forests, bagged trees, decision trees, boosted trees, or boosted stumps-may be used in different embodiments. The machine learning model 1420, when applied to the feature vector extracted from the input data item, outputs an indication of whether the input data item has the property in question, such as a Boolean yes/no estimate, or a scalar value representing a probability.

In some embodiments, a validation set is formed of additional input data, other than those in the training sets, which have already been determined to have or to lack the property in question. The machine learning system 1450 applies the trained machine learning model 1420 to the data of the validation set to quantify the accuracy of the machine learning model 1420. Common metrics applied in accuracy measurement include: Precision=TP/(TP+FP) and Recall=TP/(TP+FN), where precision is how many the machine learning model correctly predicted (TP or true positives) out of the total it predicted (TP+FP or false positives), and recall is how many the machine learning model correctly predicted (TP) out of the total number of input data items that did have the property in question (TP+FN or false negatives). The F score (F-score=2*PR/(P+R)) unifies precision and recall into a single measure. In one embodiment, the machine learning module iteratively re-trains the machine learning model until the occurrence of a stopping condition, such as the accuracy measurement indication that the model is sufficiently accurate, or a number of training rounds having taken place.

In some implementations, the machine learning model 1420 is a convolutional neural network (CNN). A CNN can be configured based on a presumption that inputs to the CNN correspond to image pixel data for an image or other data that includes features at multiple spatial locations. For example, sets of inputs can form a multi-dimensional data structure, such as a tensor, that represent color features of an example digital image (e.g., a biological image of biological tissue). In some implementations, inputs to the CNN correspond to a variety of other types of data, such as data obtained from different devices and sensors of a vehicle, point cloud data, audio data that includes certain features or raw audio at each of multiple time steps, or various types of one-dimensional or multiple dimensional data. A convolutional layer of the CNN can process the inputs to transform features of the image that are represented by inputs of the data structure. For example, the inputs are processed by performing dot product operations using input data along a given dimension of the data structure and a set of parameters for the convolutional layer.

Performing computations for a convolutional layer can include applying one or more sets of kernels to portions of inputs in the data structure. The manner in which CNN performs the computations can be based on specific properties for each layer of an example multi-layer neural network or deep neural network that supports deep neural net workloads. A deep neural network can include one or more convolutional towers (or layers) along with other computational layers. In particular, for example computer vision applications, these convolutional towers often account for a large proportion of the inference calculations that are performed. Convolutional layers of a CNN can have sets of artificial neurons that are arranged in three dimensions, a width dimension, a height dimension, and a depth dimension. The depth dimension corresponds to a third dimension of an input or activation volume and can represent respective color channels of an image. For example, input images can form an input volume of data (e.g., activations), and the volume has dimensions 32×32×3 (width, height, depth respectively). A depth dimension of 3 can correspond to the RGB color channels of red (R), green (G), and blue (B).

In general, layers of a CNN are configured to transform the three dimensional input volume (inputs) to a multi-dimensional output volume of neuron activations (activations). For example, a 3D input structure of 32×32×3 holds the raw pixel values of an example image, in this case an image of width 32, height 32, and with three color channels, R, G, B. A convolutional layer of a CNN of the machine learning model 1420 computes the output of neurons that may be connected to local regions in the input volume. Each neuron in the convolutional layer can be connected only to a local region in the input volume spatially, but to the full depth (e.g., all color channels) of the input volume. For a set of neurons at the convolutional layer, the layer computes a dot product between the parameters (weights) for the neurons and a certain region in the input volume to which the neurons are connected. This computation may result in a volume such as 32×32×12, where 12 corresponds to a number of kernels that are used for the computation. A neuron's connection to inputs of a region can have a spatial extent along the depth axis that is equal to the depth of the input volume. The spatial extent corresponds to spatial dimensions (e.g., x and y dimensions) of a kernel.

A set of kernels can have spatial characteristics that include a width and a height and that extends through a depth of the input volume. Each set of kernels for the layer is applied to one or more sets of inputs provided to the layer. That is, for each kernel or set of kernels, the machine learning model 1420 can overlay the kernel, which can be represented multi-dimensionally, over a first portion of layer inputs (e.g., that form an input volume or input tensor), which can be represented multi-dimensionally. For example, a set of kernels for a first layer of a CNN may have size 5×5×3×16, corresponding to a width of 5 pixels, a height of 5 pixel, a depth of 3 that corresponds to the color channels of the input volume to which to a kernel is being applied, and an output dimension of 16 that corresponds to a number of output channels. In this context, the set of kernels includes 16 kernels so that an output of the convolution has a depth dimension of 16.

The machine learning model 1420 can then compute a dot product from the overlapped elements. For example, the machine learning model 1420 can convolve (or slide) each kernel across the width and height of the input volume and compute dot products between the entries of the kernel and inputs for a position or region of the image. Each output value in a convolution output is the result of a dot product between a kernel and some set of inputs from an example input tensor. The dot product can result in a convolution output that corresponds to a single layer input, e.g., an activation element that has an upper-left position in the overlapped multi-dimensional space. As discussed above, a neuron of a convolutional layer can be connected to a region of the input volume that includes multiple inputs. The machine learning model 1420 can convolve each kernel over each input of an input volume. The machine learning model 1420 can perform this convolution operation by, for example, moving (or sliding) each kernel over each input in the region.

The machine learning model 1420 can move each kernel over inputs of the region based on a stride value for a given convolutional layer. For example, when the stride is set to 1, then the machine learning model 1420 can move the kernels over the region one pixel (or input) at a time. Likewise, when the stride is 2, then the machine learning model 1420 can move the kernels over the region two pixels at a time. Thus, kernels may be shifted based on a stride value for a layer and the machine learning model 1420 can repeatedly perform this process until inputs for the region have a corresponding dot product. Related to the stride value is a skip value. The skip value can identify one or more sets of inputs (2×2), in a region of the input volume, that are skipped when inputs are loaded for processing at a neural network layer. In some implementations, an input volume of pixels for an image can be “padded” with zeros, e.g., around a border region of an image. This zero-padding is used to control the spatial size of the output volumes.

As discussed previously, a convolutional layer of CNN is configured to transform a three dimensional input volume (inputs of the region) to a multi-dimensional output volume of neuron activations. For example, as the kernel is convolved over the width and height of the input volume, the machine learning model 1420 can produce a multi-dimensional activation map that includes results of convolving the kernel at one or more spatial positions based on the stride value. In some cases, increasing the stride value produces smaller output volumes of activations spatially. In some implementations, an activation can be applied to outputs of the convolution before the outputs are sent to a subsequent layer of the CNN.

An example convolutional layer can have one or more control parameters for the layer that represent properties of the layer. For example, the control parameters can include a number of kernels, K, the spatial extent of the kernels, F, the stride (or skip), S, and the amount of zero padding, P. Numerical values for these parameters, the inputs to the layer, and the parameter values of the kernel for the layer shape the computations that occur at the layer and the size of the output volume for the layer. In some implementations, the spatial size of the output volume is computed as a function of the input volume size, W, using the formula (W−F+2P)/S+1. For example, an input tensor can represent a pixel input volume of size [227×227×3]. A convolutional layer of a CNN can have a spatial extent value of F=11, a stride value of S=4, and no zero-padding (P=0). Using the above formula and a layer kernel quantity of K=116, the machine learning model 1420 performs computations for the layer that results in a convolutional layer output volume of size [55×55×156], where 55 is obtained from [(227−11+0)/4+1=55].

The computations (e.g., dot product computations) for a convolutional layer, or other layers, of a CNN involve performing mathematical operations, e.g., multiplication and addition, using a computation unit of a hardware circuit of the machine learning model 1420. The design of a hardware circuit can cause a system to be limited in its ability to fully utilize computing cells of the circuit when performing computations for layers of a neural network.

In the previous description, for the purposes of explanation, numerous specific details are set forth in order to provide a thorough understanding of the present disclosure. However, the present disclosure may be practiced without these specific details. In other instances, well-known structures and devices are shown in block diagram form in order to avoid unnecessarily obscuring the present disclosure.

In the drawings, specific arrangements or orderings of schematic elements, such as those representing devices, modules, instruction blocks and data elements, are shown for ease of description. However, it should be understood by those skilled in the art that the specific ordering or arrangement of the schematic elements in the drawings is not meant to imply that a particular order or sequence of processing, or separation of processes, is required. Further, the inclusion of a schematic element in a drawing is not meant to imply that such element is required in all embodiments or that the features represented by such element may not be included in or combined with other elements in some implementations.

Further, in the drawings, where connecting elements, such as solid or dashed lines or arrows, are used to illustrate a connection, relationship, or association between or among two or more other schematic elements, the absence of any such connecting elements is not meant to imply that no connection, relationship, or association can exist. In other words, some connections, relationships, or associations between elements are not shown in the drawings so as not to obscure the disclosure. In addition, for ease of illustration, a single connecting element is used to represent multiple connections, relationships or associations between elements. For example, where a connecting element represents a communication of signals, data, or instructions, it should be understood by those skilled in the art that such element represents one or multiple signal paths (e.g., a bus), as may be needed, to affect the communication.

Reference is made in detail to implementations, examples of which are illustrated in the accompanying drawings. In the previous description, numerous specific details are set forth in order to provide a thorough understanding of the various described implementations. However, it are apparent to one of ordinary skill in the art that the various described implementations may be practiced without these specific details. In other instances, well-known methods, procedures, components, circuits, and networks have not been described in detail so as not to unnecessarily obscure aspects of the implementations.

Several features are described that can each be used independently of one another or with any combination of other features. However, any individual feature may not address any of the problems discussed above or might only address one of the problems discussed above. Some of the problems discussed above might not be fully addressed by any of the features described in this specification. Although headings are provided, data related to a particular heading, but not found in the section having that heading, may also be found elsewhere in this description.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. For example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware-or software-based (or a combination of both hardware-and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example, LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory. A computer can also include, or be operatively coupled to, one or more mass storage devices for storing data. In some implementations, a computer can receive data from, and transfer data to, the mass storage devices including, for example, magnetic, magneto optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks. Computer readable media can also include magneto optical disks and optical memory devices and technologies including, for example, digital video disc (DVD), CD ROM, DVD+/−R, DVD-RAM, DVD-ROM, HD-DVD, and BLURAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated in, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touchscreen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that is used by the user. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back end component (for example, as a data server), or that includes a middleware component (for example, an application server). Moreover, the computing system can include a front-end component, for example, a client computer having one or both of a graphical user interface or a Web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship.

Cluster file systems can be any file system type accessible from multiple servers for read and update. Locking or consistency tracking may not be necessary since the locking of exchange file system can be done at application layer. Furthermore, Unicode data files can be different from non-Unicode data files.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

In the foregoing description, embodiments of the invention have been described with reference to numerous specific details that may vary from implementation to implementation. The description and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction. Any definitions expressly set forth herein for terms contained in such claims shall govern the meaning of such terms as used in the claims. In addition, when we use the term “further comprising” or “further including” in the foregoing description or following claims, what follows this phrase can be an additional step or entity, or a sub-step/sub-entity of a previously-recited step or entity.

Particular implementations of the subject matter have been described. Other implementations, alterations, and permutations of the described implementations are within the scope of the following claims as are apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

A number of embodiments of these systems and methods have been described. Nevertheless, it are understood that various modifications may be made without departing from the spirit and scope of this disclosure.

What is claimed is:

1. A microphysiological system, comprising:
an input chamber for introducing fluid media to a tissue culture;
a tissue culture chamber comprising an apical compartment and a basolateral compartment, the apical compartment being separated from the basolateral by the tissue culture, the tissue culture comprising apical gastrointestinal tract cells facing the apical compartment and basolateral gastrointestinal tract cells facing the basolateral compartment;
a first oxygenation chamber for apical recirculation of first fluid media in an apical fluid loop;
a second oxygenation chamber for basolateral recirculation of second fluid media in a basolateral fluid loop; and
at least one sample chamber for removing the first fluid media from the apical fluid loop or for removing the second fluid media from the basolateral fluid loop.

2. The system of claim 1, further comprising a pump configured to cause a molecular compound in the fluid media to flow through the system at a circulation flow rate to distribute the molecular compound at a particular distribution rate to the tissue culture chamber.

3. The system of claim 1, wherein the input chamber is a first input chamber for introducing a first molecular compound to the tissue culture chamber, the system further comprising:
a second input chamber for introducing a second molecular compound to the tissue culture chamber in the fluid media; and
a mixing chamber for mixing the first molecular compound and the second molecular compound in the fluid media prior to introducing the first molecular compound and the second molecular compound to the tissue culture chamber.

4. The system of claim 1, further comprising a pH sensor configured to measure a pH of the fluid media from the input chamber prior to introducing the fluid media to the tissue culture chamber.

5. The system of claim 1, further comprising a controller for controlling at least one of a flow rate of the fluid media through the tissue culture chamber and a recirculation time of the fluid media through each of the apical fluid loop and the basolateral fluid loop.

6. The system of claim 1, further comprising an oxygen sensor configured to measure oxygen levels in the fluid media in the tissue culture chamber.

7. The system of claim 1, wherein the tissue culture forms a monolayer separating the fluid media in the apical compartment and the fluid media in the basolateral compartment, the monolayer having apical-to-basal permeability and basal-to-apical permeability.

8. A method for determining pharmacokinetic (PK) properties of a molecular compound in relation to gastrointestinal tissue, the method, comprising:
inputting the molecular compound into a fluid media in an input chamber coupled to a tissue culture chamber comprising an apical compartment and a basolateral compartment that are separated by the tissue culture, the tissue culture comprising apical gastrointestinal tract cells facing the apical compartment or basolateral gastrointestinal tract cells facing the basolateral compartment;
circulating the fluid media through an apical fluid loop including a first oxygenation chamber and the apical compartment;
circulating the fluid media through a basolateral fluid loop including a second oxygenation chamber and the basolateral compartment;
sampling the fluid media in at least one of the apical fluid loop or the basolateral fluid loop; and
determining a PK property from the sampled fluid media.

9. The method of claim 8, further comprising determining PK property based on a chemical structure of the molecular compound.

10. The method of claim 8, wherein determining the PK property comprises determining at least one of a bioavailability (Fa) of the molecular compound, a clearance (CL) of the molecular compound, an absorption (Papp) of the molecular compound, and intestinal metabolism of the molecular compound.

11. The method of claim 8, further comprising determining a presence or absence, in the tissue culture, of Cytochromes P450 (CYPs) enzymes, Glutathione S-transferases (GSTs) enzymes, UDP-glucuronosyltransferase (UGTs) enzymes, superfamily of sulfotransferase (SULTs) enzymes.

12. The method of claim 8, further comprising determining a drug permeability associated with the tissue culture from the apical gastrointestinal tract cells facing the apical compartment to the basolateral compartment.

13. The method of claim 8, further comprising determining a permeability of the tissue culture from the basolateral gastrointestinal tract cells facing the basolateral compartment to the apical compartment.

14. The method of claim 8, further comprising:
training a machine learning model using the determined PK property; and
predicting a human pharmacokinetic profile, oral drug absorption, or bioavailability based on the trained machine learning model.

15. A microphysiological system, comprising:
a tissue culture chamber comprising an apical compartment and a basolateral compartment, the apical compartment being separated from the basolateral by the tissue culture;
a pump for fluid media recirculation in a fluid loop including an apical fluid loop or a basolateral fluid loop; and
an oxygenation chamber for apical recirculation of fluid media in the apical fluid loop or basolateral recirculation of the fluid media in the basolateral fluid loop;
wherein the tissue culture chamber is included in a removable insert that is removable from the fluid loop.

16. The system of claim 15, wherein the removable insert is configured for being seeded with cells prior to insertion into the fluid loop.

17. The system of claim 15, wherein the removable insert is configured for being flipped to enable a different compartment of the tissue culture chamber to be in the fluid loop.

18. The system of claim 15, further comprising:
an input chamber for introducing a first molecular compound to the tissue culture chamber;
a second input chamber for introducing a second molecular compound to the tissue culture chamber in the fluid media; and
a mixing chamber for mixing the first molecular compound and the second molecular compound in the fluid media prior to introducing the first molecular compound and the second molecular compound to the tissue culture chamber.

19. The system of claim 15, further comprising a controller for controlling at least one of a flow rate of the fluid media through the tissue culture chamber and a recirculation time of the fluid media through the fluid loop.

20. The system of claim 15, further comprising an oxygen sensor configured to measure oxygen levels in the fluid media in the tissue culture chamber.
